SEQUENCE LISTING (3 sequences)

SEQ ID NO: 1 - protein, 9 aa, synthetic construct
RRRRRRRRR

SEQ ID NO: 2 - DNA, 24 bp, synthetic construct
  source 1..24: mol_type = other DNA
ctactattttgatgagatagtaga

SEQ ID NO: 3 - DNA, 16 bp, synthetic construct
  source 1..16: mol_type = other DNA
tctactatctcatcaa

(19) United States
(12) Patent Application Publication
(10) Pub. No.: US 2025/0179558 A1
(43) Pub. Date: Jun. 5, 2025

(54) ENHANCING FLUORESCENCE BASED NUCLEOTIDE BIOSENSORS THROUGH LIQUID-LIQUID PHASE SEPARATION USING PEPTIDE-BASED COACERVATES

(71) Applicant: The Government of the United States of America, as represented by the Secretary of the Navy, Arlington, VA (US)

(72) Inventors: Sebastian A. Diaz, Silver Spring, MD (US); Deborah Sementa, New York, NY (US); Igor L. Medintz, Silver Spring, MD (US); Rein Ulijn, New York, NY (US); Christopher M. Green, Lorton, VA (US)

(73) Assignee: The Government of the United States of America, as represaented by the Secretary of the Navy, Arlington, VA (US)

(21) Appl. No.: 18/931,519

(22) Filed: Oct. 30, 2024

Related U.S. Application Data
(60) Provisional application No. 63/594,358, filed on Oct. 30, 2023.

(51) Int. Cl. C12Q 1/6818

(52) U.S. Cl. CPC C12Q 1/6818

(57) ABSTRACT

A system includes an aqueous solution containing a positively charged peptide, a molecular counterion thereto, and a molecular beacon. The molecular beacon includes a DNA construct including single-stranded DNA tending to form a hairpin (HP) configuration and having (1) a central loop region complementary to a target sequence, (2) regions at opposing ends complementary to one another, and (3) a fluorophore and a quencher thereof at the opposing ends, such that binding with the target sequence results in dissociation of the DNA regions at the opposing ends, thereby separating the fluorophore and quencher with a resulting increase in fluorescence. The system may be useful in nucleotide detection methods.

### CROSS-REFERENCE TO RELATED APPLICATIONS

This application claims priority to U.S. Provisional Patent Application No. 63/594,358, filed Oct. 30, 2023, which is incorporated by reference herein in its entirety.

### FEDERALLY SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 211760.

### INCORPORATION BY REFERENCE

This application incorporates by reference the Sequence Listing XML file submitted herewith via the patent office electronic filing system having the file name “211760.xml” and created on Oct. 29, 2024, with a file size of 4 kilobytes.

### BACKGROUND

Peptide-based liquid-liquid phase separated domains, i.e., liquid condensates or coacervates, are a new biomaterial with many exciting potential applications. They are self-forming membraneless structures where a liquid condensed phase rich in biomolecules, separates from the original aqueous solution. Coacervates are neither pure homogenous liquid phase nor a heterogenous aggregate and display varying degrees of order inside a mostly disordered system. Coacervates are μm scaled multicomponent systems that have been shown to colocalize chemical and biological moieties and materials. The continuous aqueous phase inside and outside the coacervate allows for steady exchange between the phases. Coacervates represent a growing area of research, due to their importance in subcellular organization, and potential use in designing biomimetic materials and new tools for synthetic biology. One approach taken by researchers is to utilize designer peptides to create these systems, though the design rules are only now beginning to be understood. Due to the condensed phase, need for charge equilibration, and change in hydrophobicity, coacervates are capable of sequestering and concentrating molecules from solution. This includes peptides naturally, as well as polynucleotides and enzymes.

Sensing RNA and DNA is of great importance due to the capability of these nucleotides to function as reporters of virus, bacterial, and mycotic infections; there is also a growing thought that microRNA can be utilized as biomarkers for a whole range of human diseases. The most specific probe for nucleotides are their complementary sequences, capable of discerning single nucleotide base mismatches along with single nucleotide polymorphisms (SNPs). To this end, nucleic acids molecular probes offer ease of synthesis and a range of available modifications. Specifically, a molecular beacon (MB) in the form of hairpin (HP) DNA probes, composed of single stranded DNA (ssDNA) which folds upon itself to form a stem-loop structure, are particularly useful in conjunction with fluorescent readouts. Such MBs are generally labeled with two distinct fluorescent dye species at either end. When the MB is closed the two dyes function as a donor and acceptor Förster resonance energy transfer (FRET) system. Upon binding of the target, which is complementary to the loop and part of the HP stem, the MB unfolds and becomes a partially linear double stranded DNA (dsDNA); this separates the two fluorescent tags, avoids reformation of the closed HP form and minimizes the FRET, resulting in a large change in the fluorescent peak ratios of the dyes. The MB signal change is directly correlated to the concentration of the target strand in solution. The opening of the HP is based on the interaction with the strand, which is based on the equilibrium of the MB and target strands interacting.

It would be desirable to develop new systems and methods for improving the properties of nucleotide biosensors.

### BRIEF DESCRIPTION

In one embodiment, a system comprises an aqueous solution comprising a positively-charged peptide, a molecular counterion thereto (such as adenosine triphosphate (ATP)), and a molecular beacon, wherein the molecular beacon comprises a DNA construct comprising single-stranded DNA tending to form a hairpin (HP) configuration and comprising (1) a central loop region complementary to a target sequence, (2) regions at opposing ends complementary to one another, and (3) a fluorophore and a quencher thereof at the opposing ends, such that binding with the target sequence results in dissociation of the DNA regions at the opposing ends, thereby separating the fluorophore and quencher with a resulting increase in fluorescence; and the positively-charged peptide has SEQ ID NO: 1 and is present in an amount effective to form coacervates upon interaction with the ATP which is also in the required amount thus concentrating the molecular beacon and, if present, the target sequence thereof, in the coacervates.

In another embodiment, a method of nucleotide detection comprises combining in an aqueous solution a positively-charged peptide, a molecular counterion thereto (such as ATP) and a molecular beacon, and a nucleotide comprising a target sequence of the molecular beacon; and measuring fluorescence of the fluorophore, wherein such fluorescence indicates presence of the nucleotide, wherein the molecular beacon comprises a DNA construct comprising single-stranded DNA tending to form a HP configuration and comprising (1) a central loop region complementary to the target sequence, (2) regions at opposing ends complementary to one another, and (3) a fluorophore and a quencher thereof at the opposing ends, such that binding with the target sequence results in dissociation of the DNA regions at the opposing ends, thereby separating the fluorophore and quencher with a resulting increase in fluorescence; and the positively-charged peptide has SEQ ID NO: 1 and is present in an amount effective to form coacervates upon interaction with the ATP which is also in the required amount, thus concentrating the molecular beacon and the nucleotide comprising the target sequence thereof in the coacervates.

Disclosed, in some embodiments, is a system including an aqueous solution having a positively charged peptide, a molecular counterion thereto, and a molecular beacon. The molecular beacon includes a DNA construct having single-stranded DNA tending to form a HP configuration and including (1) a central loop region complementary to a target sequence, (2) regions at opposing ends complementary to one another, and (3) a fluorophore and a quencher thereof at the opposing ends, such that binding with the target sequence results in dissociation of the DNA regions at the opposing ends, thereby separating the fluorophore and quencher with a resulting increase in fluorescence. The positively charged peptide is present in an amount effective to form coacervates thus concentrating the molecular beacon and, if present, the target sequence thereof, in the coacervates. The positively charged peptide may be SEQ ID NO: 1. In some embodiments, the molecular counterion is adenosine triphosphate (ATP). The coacervates may have an average diameter in a range of from about 1 μm to about 4 μm. In some embodiments, the positively-charged peptide includes a poly-histidine. A concentration of the polypeptide in the aqueous solution may be at least 150 μM, optionally at least 250 μM. In some embodiments, a concentration of the molecular counterion in the aqueous solution is at least 350 μM.

Disclosed, in other embodiments, is a method of nucleotide detection. The method includes combining in an aqueous solution a positively-charged peptide, a molecular counterion thereto, a molecular beacon, and a nucleotide containing a target sequence of the molecular beacon; and measuring fluorescence of the fluorophore, wherein such fluorescence indicates presence of the nucleotide, wherein the molecular beacon comprises a DNA construct comprising single-stranded DNA tending to form a HP configuration and comprising (1) a central loop region complementary to the target sequence, (2) regions at opposing ends complementary to one another, and (3) a fluorophore and a quencher thereof at the opposing ends, such that binding with the target sequence results in dissociation of the DNA regions at the opposing ends, thereby separating the fluorophore and quencher with a resulting increase in fluorescence; and the positively-charged peptide is present in an amount effective to form coacervates, thus concentrating the molecular beacon and the nucleotide comprising the target sequence thereof in the coacervates. The positively charged peptide may have SEQ ID NO: 1 or 2. In some embodiments, the molecular counterion is adenosine triphosphate (ATP). The coacervates may have an average diameter in a range of from about 1 μm to about 4 μm. In some embodiments, the positively-charged peptide includes a poly-histidine. A concentration of the polypeptide in the aqueous solution may be at least 150 μM, optionally at least 250 μM. In some embodiments, a concentration of the molecular counterion in the aqueous solution is at least 350 μM.

Disclosed, in further embodiments, is a composition for detecting a target sequence. The composition includes: water; coacervate droplets dispersed in the water; and a molecular beacon comprising a DNA construct comprising single-stranded DNA tending to form a HP configuration and comprising (1) a central loop region complementary to a target sequence, (2) regions at opposing ends complementary to one another, and (3) a fluorophore and a quencher thereof at the opposing ends, such that binding with the target sequence results in dissociation of the DNA regions at the opposing ends, thereby separating the fluorophore and quencher with a resulting increase in fluorescence. The molecular beacon and if present, the target sequence, are concentrated in the coacervate droplets. In some embodiments, the coacervate droplets are formed from a positively charged peptide having SEQ ID NO: 1 and adenosine triphosphate (ATP) as a molecular counterion. The coacervate droplets may have an average diameter in a range of from about 1 μm to about 4 μm.

These and other non-limiting aspects of the disclosure are more particularly set forth below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following is a brief description of the drawings, which are presented for the purposes of illustrating the exemplary embodiments disclosed herein and not for the purposes of limiting the same.

FIG. 1A provides a schematic of a DNA molecular beacon in hairpin (HP) and dsDNA form. The HP form places the Cy3-Cy5 FRET pair in close proximity, quenching the Cy3 and maximizing Cy5 emission. The dsDNA form separates the FRET pair, recovering Cy3 emission and lowering Cy5 signal. FIG. 1B shows the fluorescence spectra of the two forms upon 525 nm excitation. FIG. 1C shows a schematic of coacervate formation upon addition of R9 peptide and ATP counterion. After coacervate formation, addition of DNA leads to sequestration of the DNA within the coacervate. At right a fluorescence microscopy image demonstrating that the labeled-DNA has colocalized almost entirely within the coacervates.

FIG. 2A depicts Cy3/Cy5 ratio, normalized to 0 target addition at time equals 0, as a function of target and time in buffer. Circles=0.5 target DNA for each MB strand. Triangles=1.2 target DNA for each MB strand. FIG. 2B shows Cy3/Cy5 ratio, normalized to 0 target addition at time equals 0, as a function of target and time where solution contains R9-ATP coacervates. Circles=0.5 target DNA for each MB strand. Triangles=1.2 target DNA for each MB strand.

FIG. 3A shows the normalized Cy5/Cy3 ratio, with 0 target addition at time equals 0 set as 1, as a function of the ratio between target DNA and MB. Black squares=buffer; Lighter circles=coacervates. Uncertainties are determined through triplicates. Concentrations were 400 μM R9, 500 μM ATP, DNA MB 250 nM. Buffer: 10 mM Tris·HCl containing 15 mM KCl, 0.5 mM MgCl₂. FIG. 3B contains zoomed in data from FIG. 3A, showing the limit of detection (LOD). It can be seen that coacervates lower the LOD to 0.005 target: MB, while in buffer the value is between 0.1 and 0.25. Additionally, the signal-to-noise is improved more than 8-fold.

FIG. 4A provides a kinetic trace of the time required to reach maximum FRET signal change for a 1:1 target: MB ratio depending on whether the assay is done in buffer or coacervate. One consistently observes faster kinetics in the coacervate. FIG. 4B shows an increase in the fluorescence quantum yield of the dyes composing the FRET pair as a function of being in the coacervate in comparison to the same buffer. Measured both in steady-state and fluorescence lifetime format. Cy3 has a ˜3.5-fold increase in its fluorescence quantum yield. Cy5 has an ˜1.25-fold increase in fluorescence quantum yield. Taking the total fluorescence of the FRET pair, one sees a considerable increase, though with a discrepancy between the steady-state and lifetime measurement.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named components/steps and allowing the presence of other components/steps. The term “comprising” should be construed to include the term “consisting of,” which allows the presence of only the named components/steps.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

As used herein, the term “about” when used in conjunction with a stated numerical value or range denotes somewhat more or somewhat less than the stated value or range, to within a range of ±10% of that stated.

As used herein, the term “molecular beacon” refers to a DNA construct comprising single-stranded DNA tending to form a hairpin (HP) configuration and comprising (1) a central loop region complementary to a separate target sequence, (2) regions at opposing ends complementary to one another, and (3) a fluorophore and a quencher thereof at the opposing ends, such that binding with the target sequence results in dissociation of the DNA regions at the opposing ends, thereby separating the fluorophore and quencher with a resulting increase in fluorescence.

As described herein, coacervates offer a simple and biocompatible way to increase the sensitivity of nucleic acid HP probe biosensors, also known as molecular beacons, as they concentrate both the sensor constructs and their target strands within the coacervate. This results in localized higher concentrations and therefore increased sensitivity. Experiments have found a 50-fold lowering of the limit of detection within coacervates when compared to buffer solutions. Further benefits include an increase in the kinetics with the final equilibrium stage being reached more than 4-times faster in coacervates and an increase in the dye fluorescent quantum yields within the coacervates, resulting in greater signal to noise ratios.

Peptide-based in vitro liquid-liquid phase separated domains, i.e., liquid condensates, biomolecular condensates, or coacervates, are inspired by membraneless organelles with important regulatory functions in biological systems. Coacervates as a de novo biomaterial has become a new focus due to the many exciting potential applications in fields ranging from biosensing to drug delivery. Through these micrometer scaled self-forming membraneless structures, a liquid condensed phase rich in biomolecules separates itself from the original aqueous solution. Coacervates are neither pure homogenous liquid phases nor heterogeneous aggregates, displaying varying degrees of supramolecular order inside a mostly disordered system. The continuous aqueous environment inside and surrounding the coacervate enables steady exchange between these phases, a crucial factor when biosensing is the application of interest. Due to the condensed phase's need for charge equilibration, along with its modified hydrophobicity and dielectric constant, coacervates are capable of sequestering and concentrating molecules from solution. In this context, simple peptide derived coacervates have been reported to successfully recruit nucleotides into the condensate phase, similarly to proteins that often enlist DNA or RNA within membraneless compartments inside the cells.

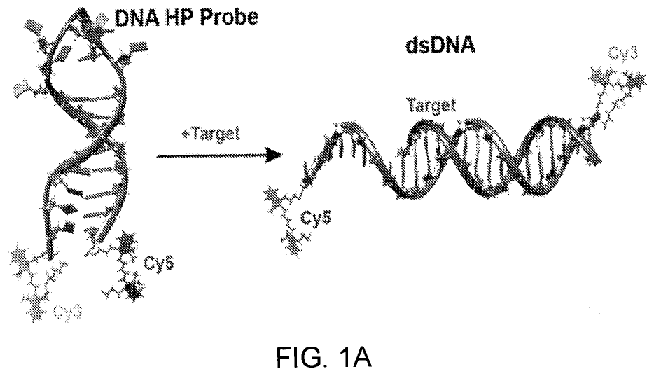

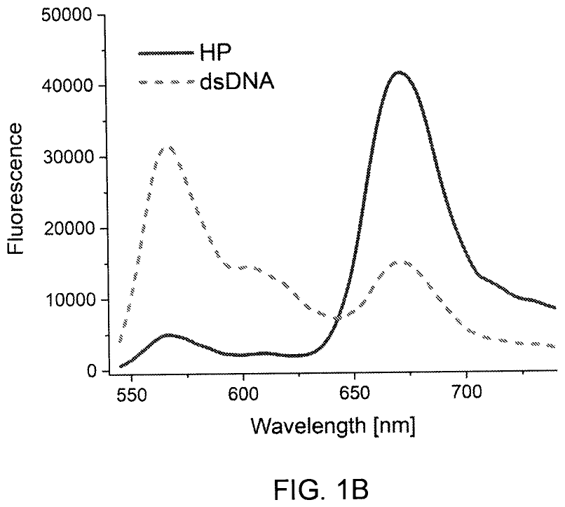

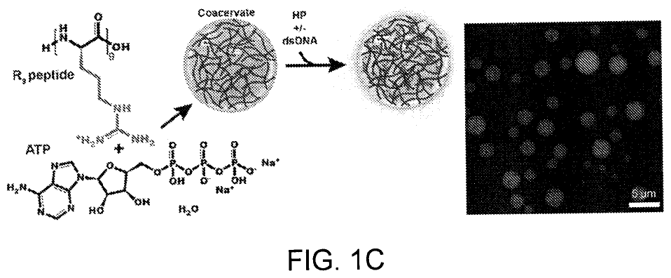

Coacervates are a minimalist and biocompatible way to increase the sensitivity of nucleotide biosensors as they can concentrate both the MB and target strands within the coacervate. The polyarginine peptide, specifically R9, as a positively charged component, with adenosine triphosphate (ATP), as the negatively charged counterpart that co-assemble to form liquid droplets. At concentrations greater than 150 μM of the polyarginine peptide and 350 μM of ATP, coacervates form with diameters ranging between 1-4 μm. These coacervates efficiently capture the majority of the DNA-based MB, as verified by fluorescence microscopy (FIG. 1C).

The change in the fluorescence peak ratios of the MB is directly correlated to the distribution between the HP and dsDNA forms, which is a function of the concentration of the target strand in solution. The oligonucleotide sequestration within the coacervates resulted in higher localized concentrations and therefore increased sensitivity. Experiments have found a greater than 20-fold lowering of the limit of detection (LOD) within coacervates when compared to reactions in bulk buffer solutions. Furthermore, additional benefits of utilizing coacervates include: (i) an increase in the sensing kinetics with the final equilibrium stage being reached more than 4-times faster in coacervates; and (ii) an increase in the dye fluorescent quantum yields (QYs) within the coacervates, resulting in greater signal-to-noise (S/N) ratios.

The poly(arginine) peptide, specifically R9 (SEQ ID No: 1) or H12 (SEQ ID No: 2), which are very positively charged at physiological pHs, can be exploited to create coacervates in conjunction with adenosine triphosphate (ATP). It was seen that other similarly charged peptides of similar length, such as poly-histidines, could also operate in this way. Peptide-based coacervates can concentrate nucleotides such as DNA and RNA into the condensate phase. At concentrations greater than 250 μM of the peptide and 350 μM of ATP, the coacervates form and will sequester the majority of the DNA (FIG. 1A and FIG. 1B), which was confirmed through microscopy and centrifugation experiments. It is expected that other suitable counterions could substitute for ATP, including adenosine diphosphate (ADP), adenosine monophosphate (AMP), as well as 4-(2-Hydroxyethyl) piperazine-1-ethane-sulfonic acid (HEPES).

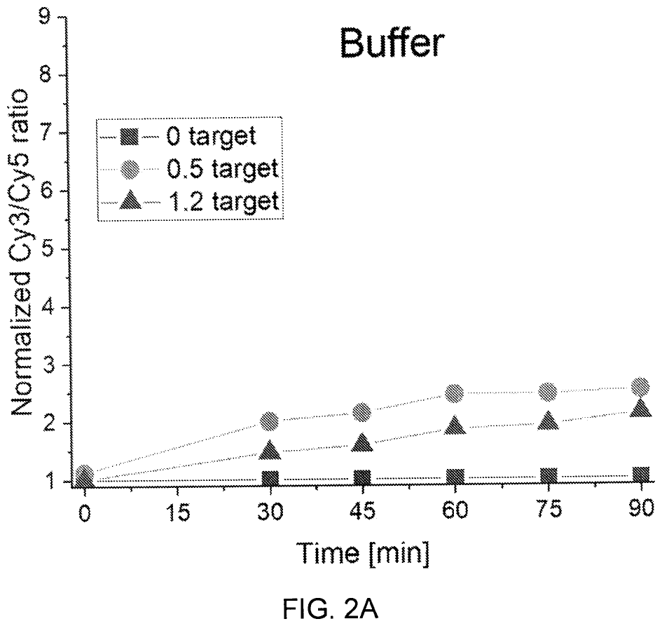

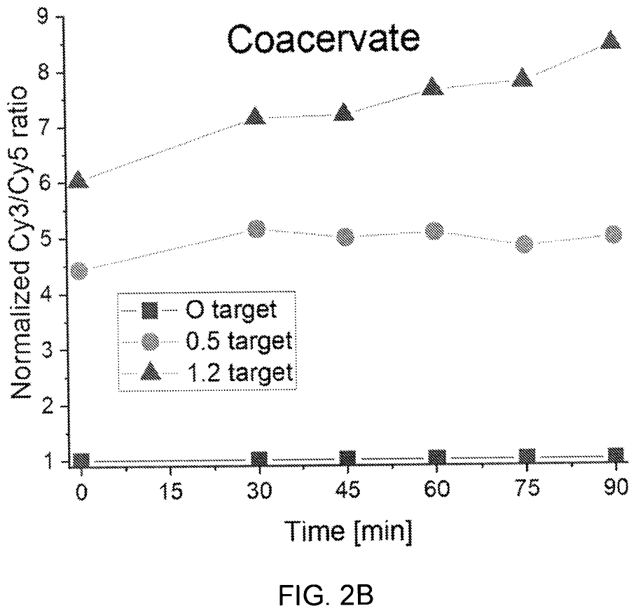

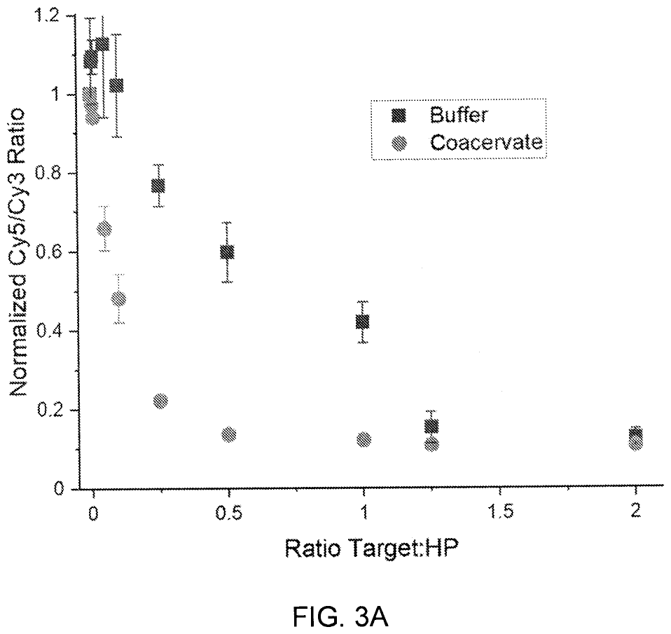

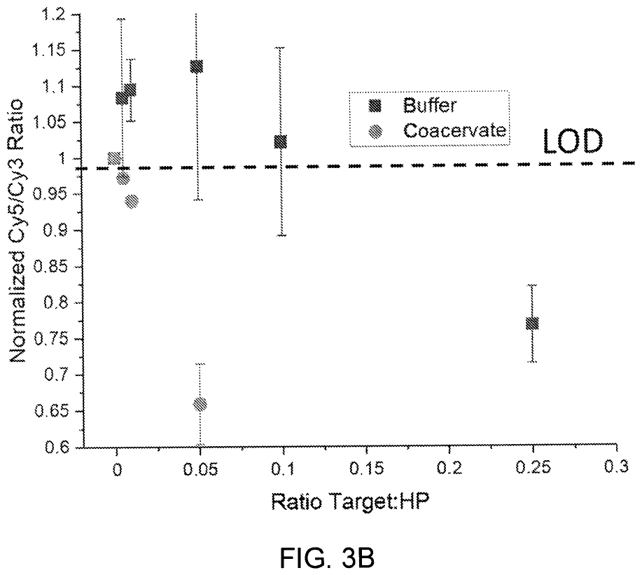

Peptide coacervates self-assemble in common aqueous buffers into 1-5 μm diameter membraneless organelles and then sequester nucleotides (FIG. 1A and FIG. 1B). This results in localized higher nucleotide concentrations and therefore increased sensitivity (FIGS. 2A and 2B). Using 400 μM R9 peptide, 500 μM ATP, DNA HP 250 nM. Buffer was 10 mM Tris·HCl containing 15 mM KCl, 0.5 mM MgCl₂. Experiments have found a 50-fold lowering of the limit of detection within coacervates when compared to buffer solutions (FIGS. 3A and 3B).

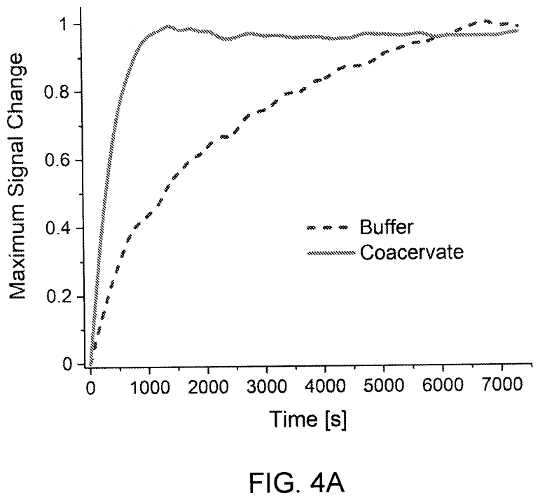

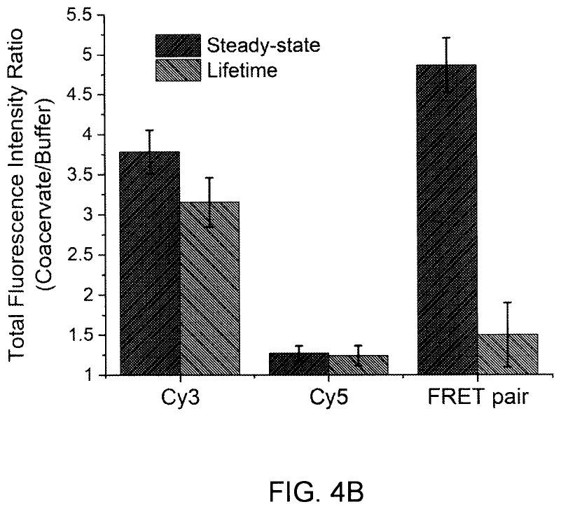

Furthermore, additional benefits included an increase in the kinetics with the maximum change in signal being reached at least 2-times faster in coacervates (FIG. 4A) and an increase in the dye fluorescent quantum yields within the coacervates (FIG. 4B), resulting in greater signal to noise ratios. Other nucleotide analogs, such as peptide nucleic acids were also found to localize to the coacervates. Alternative peptides, such as poly histidine peptides, were also evaluated and resulted in coacervate formation with localization of dye labeled DNA to the formed coacervates. Furthermore, DNA localized within coacervates was less susceptible to common nucleases such as DNase, Exo I, Exo III, and restriction enzymes such as EcoR1.

The tested HP probe and target sequences are seen below in Table 1. The molecular beacon includes a Cy5 at the 5′ end and a Cy3 at the 3′ end.

TABLE 1
DNA strands

| Strand | Sequence (5′→3′) |
|---|---|
| MB | /5Cy5/CTACTATTTTGATGAGATAGTAGA/3Cy3Sp/ |
| | (SEQ ID No: 3) |
| Target | TCTACTATCTCATCAA (SEQ ID No: 4) |

This technique offers at least the following advantages:

(1) The ability to increase the concentrations of natural oligonucleotides (DNA and RNA).

(2) The ability to increase the concentrations of oligonucleotide analogs (Locked nucleic acid, Peptide nucleic acid, etc.).

(3) The ability to increase the kinetics of DNA interactions, specifically DNA complementary binding and displacement reactions.

(4) The ability to improve the sensitivity and lower the limit of detection of nucleotide based biosensors.

(5) The ability to enhance the fluorescence quantum yield of organic dyes within coacervates.

(6) The ability to increase the FRET efficiency of dye pairs within coacervates.

(7) The ability to increase the signal to noise ratio of FRET based sensors within coacervates.

(8) The ability to increase the speed and efficiency of DNA computing approaches based on DNA toe-hold displacement.

(9) The ability to utilize the coacervate to stabilize the nucleotides, showing resistance to most common nucleases for long-term use.

(10) The ability to capture natural oligonucleotides (DNA and RNA).

(11) The ability to capture and increase the concentration of nanoparticles and other soft and hard nanomaterials.

(12) The ability to capture and increase the concentration of nanoparticles and other soft and hard nanomaterials that are bioconjugated with biomaterials.

(13) The ability to capture and increase the concentration of nanoparticle-based biosensors and improve their activity in a similar manner.

Sequestration within peptide coacervates improves the fluorescence intensity, kinetics, and limits of detection of dye-based DNA biosensors.

The use of coacervates as a medium for increased functionality of oligonucleotide-based biosensors. The functionality of DNA biosensors is not only preserved within coacervates, but they also worked at much lower concentrations and proportions of target strand to MB strand, due to the droplets capability to sequester oligonucleotides, raising their local concentration more than 100-fold. It is also possible that the R9 destabilizes the MB allowing for a lower energy barrier for transition to the dsDNA form, improving kinetics and sensitivity. Other benefits included the improved S/N due to the increased fluorescence intensity of the dyes and enhanced kinetics. Though the full cause of the increased kinetics is not known at this time; the local increase in concentration maximizing the collision frequency, and R9 destabilization of the MB, may play the two largest roles.

It is believed that even greater enhancement could still be achieved. For example, the designed MB could be improved by avoiding the quenching of the Cy5 in the HP form and by extending the dsDNA form to lower the FRET in the open form and maximize the Cy3 signal. This would increase the dynamic range of the sensor by creating a much greater change in the ratio of Cy5/Cy3 fluorescence and would likely improve the LOD even further. It is also possible that further optimization of the polypeptide chosen for the coacervate could lead to greater sequestration (lower LOD), smaller coacervates (faster kinetics), and more viscous interiors (improved S/N). As seen in the initial experiments, the MB can already have interacted with the target and subsequently integrate into the coacervate to improve the signal, this means that the order of addition is not important, which should simplify sample preparation for biosensing applications. Beyond biosensing, DNA systems are being postulated as possible next-generation data storage and computing hardware but have been challenged by low retrieval and reading speed. To date most of the approaches to realize these applications utilize similar recognition and displacement approaches as shown within this manuscript, it thus seems conceivable that coacervates will provide a way to improve the speed and minimize the leakiness of these systems.

The following examples are provided to illustrate the systems and methods of the present disclosure. The examples are merely illustrative and are not intended to limit the disclosure to the materials, conditions, or process parameters set forth therein.

### Examples

### Materials and Methods

R9 peptide (used after trifluoroacetic acid, TFA, removal) and DNA were purchased from Genscript and IDT, respectively. ATP (CAS: 34369-07-8) and salts for buffers purchased from Sigma Aldrich and used as is. The utilized buffer was 10 mM Tris·HCl, 15 mM KCl, 0.5 mM MgCl₂, pH 7.6.

### TFA Removal

Peptides were dissolved in a 50 mM HCl solution to a concentration of 1 mg/ml. The solutions were sonicated for 5 min and lyophilized. The procedure was repeated 5 times.

### Slide Surface Functionalization

Glass coverslips were surface coated following the method previously described. Briefly, cover slides were treated with 0.5 M KOH in iso-propanol for 30 min, washed and dried overnight at 90° C. Subsequent silanization was carried out using a 3 mg/ml solution of N-(Triethoxysilylpropyl)-O-polyethylene oxide urethane in toluene for 4 h. Slides were dried and wiped to remove any evaporation-induced marks. Functionalized slides were used for fluorescence imaging as it allows the condensates to adopt more spherical shapes.

### Sample Preparation

Coacervates were formed by creating stock solutions of R9, ATP, and the required DNA, along with the buffer solution. R9 was added to the buffer in an Eppendorf tube and then ATP was added subsequently and mixed through pipetting. In general the MB, or the pre-formed MB+target, was added and allowed to sequester for 20 min at room temperature. For kinetics experiments, the solution containing the MB, either buffer or coacervate, was added to a fluorescence 384-microwell plate and then the target was added to each well using a multipipetter.

### Absorption Spectra

Spectra were measured using a Cary 60 UV-Vis spectrophotometer for all samples. Measurements were performed within the spectral range of 200-900 nm, with 1 nm step intervals and a 0.1 sec integration time in a 10 mm path length quartz spectrophotometer cuvette was used for the measurements. Background scattering was corrected for using the OriginLab PeakAnalyzer function in the case of the coacervates samples.

### Fluorescence Spectra

Spectra were measured at 20° C. using a TECAN Spark plate reader exciting from above on a 384-microwell plate. An excitation wavelength of 520 nm was used to excite the sample and the fluorescence emission was measured from 540-725 nm with 5 nm steps when collecting the entire spectra, or the Cy3 and Cy5 peaks were followed at 570 and 670 nm, respectively.

### Confocal Fluorescence Microscopy

Imagining was performed as reported previously. Briefly, samples were imaged on functionalized glass slide-coverslip chambers. 20 μl of sample was deposited in the chamber hole and covered with the coverslip. Imaging was performed using a Leica TCS SP8 STED ×3 with a ×60 objective lens (with oil immersion). Excitation was 520 nm with either the whole emission range collected (540-740 nm) for total intensity images or 10 nm windows, in the same range, were collected for spectral determinations.

### FRAP Experiments

Measurements were performed as reported previously. Briefly, Imaging was realized on a Marianas Spinning Disk confocal microscope (Intelligent Imaging Innovations) on a Zeiss Axio Observer inverted microscope using a ×100/1.46 NA PlanApochromat oil immersion objective. An area with radius=0.5 μm was bleached for 5 ms with the 488-nm line from a solid state laser (LaserStack). Subsequent recovery of the bleached area was recorded with excitation from the 488-nm laser line and collected with a 440/521/607/700-nm quad emission dichroic and 525/30-nm emission filter. Images were acquired with a Prime sCMOS camera (Photometrics) controlled by SlideBook 6 (Intelligent Imaging Innovations).

### Coacervate Design

To test a proof-of-concept system, the polyarginine (R9)-ATP coacervate was used self-assembled in a low ionic strength buffer of 10 mM Tris·HCl, 15 mM KCl, 0.5 mM MgCl₂, pH 7.6 buffer. Unless otherwise noted, the R9 was added to the buffer (final concentration 400 μM), followed by ATP (final concentration 750 μM); the consequent macroscopically observable cloudiness confirmed the formation of the coacervates. Perfect charge balance is not achieved through the macromolecules with this mixture. The charge was intentionally kept relatively positive (˜4+: 3−) to maximize DNA sequestration, though counterions will balance the overall net charge of the phases. The DNA MB was then added (250 nM final concentration) to the liquid system and left to equilibrate for 20 min at RT (20° C.) before measurements were undertaken.

### MB Design and Characterization

As an initial investigation, the nucleotide biosensor has been based on a simple design of a double-labeled DNA HP MB that senses a target strand (Table 1). The melting temperature of the HP form of the MB was determined to be 53±1° C. in the buffer (coacervate values were not determined as the coacervate is not stable at these higher temperatures), while the dsDNA was determined to be 48±2° C. While working consistently at 20° C., it was ensured that the initial structure was stable and that upon binding of the target it would not subsequently be released.

It is important to have an understanding of the predicted fluorescent changes in the MB system, and to that end, the parameters of the MB DNA as well as the FRET pair were considered. The hybridized MB: target pair would have 16 base pair (bp) in dsDNA form (5.4 nm); the full length of the MB is 24 bp plus the dye linker lengths, so it could be expected separations between the dyes, r_{DA}, of up to ˜9 nm if it was fully linear. The FRET efficiency (E_{FRET}) would depend on the dye properties, particularly the change in Forster distance, R₀, as a function of shifts in spectral overlaps and fluorescent QYs. In Table 2, QY values for the dyes in the HP or in the linear (dsDNA) format are provided, as well as in the coacervate, and compared to the buffer alone.

TABLE 2
Fluorescence quantum yield of the dyes on the MB varying confirmations
(HP and dsDNA) and conditions (buffer and coacervate)

| | Buffer | | Coacervate | |
|---|---|---|---|---|
| | HP | dsDNA | HP | dsDNA |
| Cy3 donor | 0.199 ± 0.01 | 0.16 ± 0.01 | 0.52 ± 0.04 | 0.62 ± 0.04 |
| Cy5 acceptor | 0.20 ± 0.01 | 0.28 ± 0.01 | 0.39 ± 0.03 | 0.36 ± 0.02 |

When the MB is in the HP form, the dyes are in close proximity, leading to high E_{FRET}; the dyes are, in fact, vibronically coupled and interacting strongly, as suggested by the shifts in the absorbance spectra, and thus ET saturation can be assumed. This strong coupling has been repeatedly shown to lower the fluorescence QY of cyanine dyes, including of heterodimer pairs as in this case. The proximity of specific nucleotides can also have an effect, as seen in the different dye QYs in the HP and dsDNA forms in Table 2. It is interesting to note that there is a shift and redistribution of the absorption bands in HP form when comparing the buffer to coacervate. This suggests the dyes are in a different dielectric and polar environment as well as likely change their relative position/orientation substantially in the coacervate, which could be due to the complex phase diagrams of the coacervates. Due to their strong coupling in the HP form, FRET assumptions are likely invalid, therefore the R₀ values are more relevant for the FRET pair in the dsDNA form. For buffer the R₀ value is 5.5±0.3 nm, while within coacervates it is 6.8±0.3 nm, the increase being principally due to the increased QY of the Cy3. Estimating the r_{DA} based on the physical properties of the DNA, a range of 6-9 nm would be reasonable. This broad range arises from the fact that even upon hybridization with the target, part of the MB remains as ssDNA, and that the dye linkers are very flexible. Considering the fluorescence spectra of the MB in both states (HP and dsDNA) as well as the individual dye components, the Cy3 quenching went from 95% to 45% in buffer conditions and from 96% to 32% within coacervates. Using the steady-state spectra to determine the E_{FRET} based on the Cy3 quenching, we confirmed that the experimental r_{DA} in the dsDNA con-formation was 5.6±0.5 nm in the buffer and 7.7±0.6 nm in the coacervate, which are both values within the predicted distances. This is important as it confirms that the MB is undergoing the expected conformational changes both in the buffer solution and in the more viscous and complex coacervate interior. Unexpectedly, the Cy5 signal increased upon going from the HP to dsDNA form. This is due to the above-mentioned self-quenching of Cy5 by the vibronic coupling in the HP form. Therefore, though the E_{FRET} is greatly reduced going from HP to dsDNA form, the fluorescence QY of both dyes improves to compensate for this issue. Nevertheless, the current MB design still functions for the proof-of-concept study while future MBs can alternatively be designed to avoid this issue.

### MB Properties Upon Coacervate Sequestration

Fluorescence microscopy confirmed the recruitment of the DNA biosensor inside the liquid peptide coacervates. For these experiments, either the MB by itself was added or the dsDNA form of the sensor (target strand was added in a 1:2 ratio of MB:target, the excess target assures that all the MB was in the dsDNA form), previously combined and annealed. It was observed that the fluorescence was almost fully localized within the coacervate droplets (>99%, estimated by comparing coacervate fluorescence intensity to background intensity), spectral detection showed the expected Cy5/Cy3 ratio. Then, the fact that coacervates can be concentrated through centrifugation was exploited. MB was added to the coacervates and then subsequently the sample was centrifuged (10 min at 27 k RCF), supernatant fluorescence compared to an uncentrifuged sample was less than 0.3±0.1%, while an equivalent experiment in just buffer showed no decrease in fluorescence. Through these two experiments, it was confirmed that more than 99% of the DNA was sequestered into the coacervates. Additionally, fluorescence recovery after photobleaching (FRAP) experiments corroborated the droplets' dynamic nature, independently of the DNA being in the HP or dsDNA form.

A higher fluorescence QY of the dyes within the coacervates was observed, resulting in a stronger signal from the MB (Table 2). In addition, fluorescence lifetime (T) analysis validated the steady-state results. Specifically, the Cy3 has the greatest increase in QY and lifetime (˜2.6-fold) while the Cy5 has only a slight increase (˜25%). This enhancement seems to arise from the greater viscosity found within the coacervates, which possibly limits the rotation of the methine bridge of the cyanine dyes, a common non-radiative decay mechanism.

Biosensing within Coacervates

The MB capability to function as a biosensor within the coacervates was tested. In contrast to the data presented so far, where the HP and target DNA were combined before adding them to the coacervate or buffer solution, in subsequent experiments the MB was added to the coacervate and then afterwards the target strand was added to the reaction volume, exploiting in this way the dynamic interface of the coacervates. Control experiments with a random DNA sequence added instead of the target resulted in no change in the MB fluorescence.

With the aim of testing the effect of individual coacervate components on the ability to interact with the MB sensors, an experiment was conducted where only one of the two elements, either the R9 or the ATP, was added to the buffer solution and then the MB was added. While the ATP only had no effect, the R9 appeared to interact with the MB and precipitate or quench the dye-labeled DNA, due to charge interactions. Thus, keeping constant the ATP concentration at 800 μM, the amount of R9 added was modified, with the lowest amounts being below the critical concentration levels needed to form the coacervate. Precisely, under 250 μM the peptide quenches/precipitates the MB (mechanism unclear at this time, though electrostatics is likely driving the interaction). Starting at that concentration, coacervate formation and a recovery and then improvement of the fluorescence signal occurred. The particular ratio of peptide to counterion is dependent on the choice of the two components, as well as the buffer conditions, and can vary considerably.

One interesting observation was that, not only was the signal stronger, but it appeared to reach the final state much faster within coacervates. FIG. 4A shows a representative time trace of the signal change of the MB recognizing the target strand, these experiments were realized following only the peak intensities allowing for much denser surveying of the initial time points. The y-axis has been normalized to the total fluorescence ratio change of each sample and even still, the coacervate sample has a ˜4-times shorter response time. Though kinetics were consistently faster for the coacervate, the relative difference was variable. In order to compare kinetics, seven different experiments were conducted and an increase in speed ranged from 2 to 6.5 times faster in the coacervates, with a calculated average increment of 4.4±1.7 times faster. Regarding this, slight variations in concentration, ambient temperature, mixing, and other variables that could modify solution viscosity or coacervate diameters, for example, may be responsible for this variation.

To confirm that the target DNA was being taken in by the coacervates containing the MB, experiments from the plate reader were replicated on the microscope. Coacervates were added to a functionalized slide in order to reduce their coalescence on a glass surface and imaged using 520 nm excitation. The spectral information obtained from the liquid droplets showed the expected small Cy3 emission and large Cy5 emission. The target strand was then added in twofold excess to the MB and continued to image the coacervates. As expected, the fluorescence ratio began to shift towards the Cy3 due to the switch from the HP to the dsDNA form. It was observed that at approximately 20 min after addition the reaction reached its maximal signal.

Neither improvement in S/N due to the enhanced fluorescence nor the improved kinetics that the coacervates would supply were initially expected. Due to the sequestration of the DNA and the increased local concentration, it was believed the sensitivity of nucleotide biosensors would be improved. Simple mass estimates based on solution concentrations predicted that the coacervates (the R9, ATP, and DNA) composed <0.1% of the solution mass. Even assuming this increases an order of magnitude when considering solution volume, the DNA was concentrated 100-fold, which would be on the low end of the 40000-fold previously reported for work on a more complex polymer-oligopeptide hybrid). To evaluate this, triplicate measures of the Cy5/Cy3 ratio as a function of the amount of target strand added to the MB were realized. The MB within coacervates worked much better, i.e., smaller Cy5/Cy3 ratio, than the buffer only system. In fact, using the classical LOD estimate based on 30 of a blank (0 target added) which resulted in the LOD being a ratio change below 0.97, the coacervate could detect down to 0.005 target per HP, while the buffer only system had a LOD between 0.1 and 0.25 target per HP (FIG. 3B). This is a greater than 20-fold increase in sensitivity.

The coacervate system is much more sensitive, and importantly due to the increased S/N, is able to distinguish between small amounts of target added, down to 0.5% of the MB concentration. The buffer system is much noisier (estimates from the data provide an ˜8-fold increase in uncertainty), and in fact due to the changes in QYs, the initial addition of target appears to actually increase the Cy5/Cy3 ratio, only somewhere between the 0.1 and 0.25 target: MB ratio does the change in fluorescence fall below the LOD. The coacervate system does saturate the signal change sooner, signifying that the buffer system might be preferable for distinguishing between higher amounts of target strand.

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A system comprising:
an aqueous solution comprising a positively charged peptide, a molecular counterion thereto, and a molecular beacon, wherein
the molecular beacon comprises a DNA construct comprising single-stranded DNA tending to form a hairpin (HP) configuration and comprising (1) a central loop region complementary to a target sequence, (2) regions at opposing ends complementary to one another, and (3) a fluorophore and a quencher thereof at the opposing ends, such that binding with the target sequence results in dissociation of the DNA regions at the opposing ends, thereby separating the fluorophore and quencher with a resulting increase in fluorescence; and
the positively charged peptide is present in an amount effective to form coacervates thus concentrating the molecular beacon and, if present, the target sequence thereof, in the coacervates.

2. The system of claim 1, wherein said positively charged peptide has SEQ ID NO: 1 or 2.

3. The system of claim 2, wherein said molecular counterion is adenosine triphosphate (ATP).

4. The system of claim 1, wherein said molecular counterion is adenosine triphosphate (ATP).

5. The system of claim 1, wherein the coacervates have an average diameter in a range of from about 1 μm to about 4 μm.

6. The system of claim 1, wherein the positively charged peptide comprises a poly-histidine.

7. The system of claim 1, wherein a concentration of the polypeptide in the aqueous solution is at least 150 μM.

8. The system of claim 1, wherein a concentration of the molecular counterion in the aqueous solution is at least 350 μM.

9. A method of nucleotide detection comprising:
combining in an aqueous solution a positively charged peptide, a molecular counterion thereto, a molecular beacon, and a nucleotide comprising a target sequence of the molecular beacon; and
measuring fluorescence of the fluorophore, wherein such fluorescence indicates presence of the nucleotide, wherein the molecular beacon comprises a DNA construct comprising single-stranded DNA tending to form a HP configuration and comprising (1) a central loop region complementary to the target sequence, (2) regions at opposing ends complementary to one another, and (3) a fluorophore and a quencher thereof at the opposing ends, such that binding with the target sequence results in dissociation of the DNA regions at the opposing ends, thereby separating the fluorophore and quencher with a resulting increase in fluorescence; and
the positively charged peptide is present in an amount effective to form coacervates, thus concentrating the molecular beacon and the nucleotide comprising the target sequence thereof in the coacervates.

10. The method of claim 9, wherein said positively charged peptide has SEQ ID NO: 1 or 2.

11. The method of claim 10, wherein said molecular counterion is adenosine triphosphate (ATP).

12. The method of claim 10, wherein said molecular counterion is adenosine triphosphate (ATP).

13. The method of claim 9, wherein the coacervates have an average diameter in a range of from about 1 μm to about 4 μm.

14. The method of claim 9, wherein the positively charged peptide comprises a poly-histidine.

15. The method of claim 9, wherein a concentration of the polypeptide in the aqueous solution is at least 150 μM.

16. The method of claim 9, wherein a concentration of the molecular counterion in the aqueous solution is at least 350 μM.

17. A composition for detecting a target sequence, the composition comprising:
water;
coacervate droplets dispersed in the water; and
a molecular beacon comprising a DNA construct comprising single-stranded DNA tending to form a HP configuration and comprising (1) a central loop region complementary to a target sequence, (2) regions at opposing ends complementary to one another, and (3) a fluorophore and a quencher thereof at the opposing ends, such that binding with the target sequence results in dissociation of the DNA regions at the opposing ends, thereby separating the fluorophore and quencher with a resulting increase in fluorescence;
wherein the molecular beacon and if present, the target sequence, are concentrated in the coacervate droplets.

18. The composition of claim 17, wherein the coacervate droplets are formed from a positively charged peptide having SEQ ID NO: 1 or 2 and adenosine triphosphate (ATP) as a molecular counterion.

19. The composition of claim 17, wherein the coacervate droplets have an average diameter in a range of from about 1 μm to about 4 μm.

20. The composition of claim 17, wherein the coacervate droplets are formed from a positively charged peptide having SEQ ID NO: 1 or 2 and adenosine triphosphate (ATP) as a molecular counterion; and wherein the coacervate droplets have an average diameter in a range of from about 1 μm to about 4 μm.
